(19) United States
(12) Patent Application Publication
(10) Pub. No.: US 2016/0340748 A1
(43) Pub. Date: Nov. 24, 2016

(54) MARKERS FOR DISEASE RESISTANCE IN MAIZE

(71) Applicants: UNIVERSITY OF PRETORIA, Pretoria (ZA); PANNAR SEED (PTY) LTD, Greytown (ZA)

(72) Inventors: David K. Berger, Pretoria (ZA); Maryke Carstens, Wapadrand (ZA); Fredrick J. Kloppers, Kranskop (ZA); Jacquline Meyer, Cape Town (ZA); Shane L. Murray, Lakeside (ZA); Alexander A. Myburg, Pretoria (ZA)

(21) Appl. No.: 15/106,673

(22) PCT Filed: Dec. 19, 2014
(86) PCT No.: PCT/US2014/071636

§ 371 Date: Jun. 20, 2016

(30) Foreign Application Priority Data
Dec. 20, 2013 (ZA) 2013-09651

(51) Int. Cl. C12Q 1/68

(52) U.S. Cl. CPC C12Q 1/6895; C12Q 2600/156

(57) ABSTRACT

Methods and compositions for identifying maize plants that have newly conferred tolerance or enhanced tolerance to, or are susceptible to, Gray Leaf Spot (GLS) are provided. The methods use molecular genetic markers to identify, select and/or construct tolerant plants or identify and counter-select susceptible plants. Maize plants that display newly conferred tolerance or enhanced tolerance to GLS that are generated by the methods are also a feature of the invention.

### CROSS REFERENCE TO RELATED APPLICATIONS

This application claims the benefit of South African Provisional Application No. 2013/09651, filed Dec. 20, 2013, which is incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 20141212_BB2476PCT_SequenceListing created on Dec. 12, 2014 and having a size of 14 kilobytes and is filed concurrently with the specification. The sequence listing contained in this ASCII formatted document is part of the specification and is herein incorporated by reference in its entirety.

### FIELD

The present disclosure relates to compositions and methods useful in enhancing resistance to gray leaf spot in maize plants.

### BACKGROUND

Maize is one of the most important food sources for humans and animals. Many environmental stress factors affect maize plants, impacting maize production and availability. For example, maize crops are often severely affected by gray leaf spot (GLS) caused by the fungal pathogen Cercospora zeae-maydis or Cercospora zeina (herein referred to as Cercospora spp.).

GLS is a global problem with prevalence in Africa; North, Central and South America; and Asia. Cercospora spp. overwinters in field debris and requires moisture, usually in the form of heavy fog, dew, or rain, to spread its spores and infect maize. Cercospora spp. infection in maize elicits an increased allocation of the plant's resources to protect against damaged leaf tissue, leading to elevated risk of root and stalk rot, and reduced allocation of resources to grain filling, which ultimately results in even greater crop losses. Symptoms typically include elongated, grey coloured lesions of about 1-3 mm in width and ranging from 5 to 70 mm in length occurring on leaf material. Lesions have also been noted to occur on stems during severe cases of infection. Furthermore, Cercospora spp. infection reduces grain yield and silage quality. GLS may result in yield loss of up to 68%. Therefore, reduction of the susceptibility of maize to GLS is understandably of importance.

Some commonly used GLS control methods are fungicides, crop rotation, tillage and field sanitation. Some of the disadvantages of these methods are that they are relatively expensive, ineffective or harmful to the environment. However, the most effective and most preferred method of control for GLS is the planting of resistant hybrids.

The use of phenotypic selection to introgress the GLS trait from a resistant variety into a susceptible variety can be time consuming and difficult. GLS is sensitive to environmental conditions and requires high humidity and extended leaf wetness. This sensitivity makes it difficult to reliably select for resistance to GLS from year to year based solely on phenotype (Lehmensiek et al., Theor. Appl. Genet. 103:797-803 (2001)). Specialized disease screening sites can be costly to operate, and plants must be grown to maturity in order to classify the level of resistance.

Selection through the use of molecular markers associated with GLS resistance has the advantage of permitting at least some selection based solely on the genetic composition of the progeny. Thus, GLS resistance can be measured very early on in the plant life cycle, even as early as the seed stage. The increased rate of selection that can be obtained through the use of molecular markers associated with the GLS resistance trait means that plant breeding for GLS resistance can occur at a faster rate and that commercially acceptable GLS resistant plants can be developed more quickly.

US2010/0146657 discloses a method of introgressing an allele into a maize plant including the steps of:
crossing at least one GLS resistant maize plant with at least one GLS sensitive maize plant in order to form a segregating population; and
screening said segregating population with one or more nucleic acid markers to determine if one or more maize plants from the segregating population contains a GLS resistant allele.

Furthermore, U.S. Pat. No. 5,574,210 discloses a method for the production of an inbred maize plant adapted for conferring, in hybrid combination with a suitable second inbred, resistance to GLS including the steps of:
selecting a first donor parental line possessing the desired GLS resistance having at least two of the resistant loci and crossing same with a second parental line, which is high yielding in hybrid combination, to produce a segregating plant population;
screening the plant population for identified chromosomal loci of one or more genes associated with the resistance to the GLS trait; and
selecting plants from said population having said identified chromosomal loci for further screening until a line is obtained which is homozygous for resistance to GLS at sufficient loci to give resistance to GLS in hybrid combination.

However, some of the disadvantages of the methods disclosed in US2010/0146657 and U.S. Pat. No. 5,574,210 are that few of these lines, if any, could be classified as having high resistance to GLS and that the resolution of the genetic mapping is low and therefore the markers are not tightly linked to the GLS resistance loci, which limits the applications in marker assisted breeding. Another disadvantage of these methods is that they have been tested in North and South America in conditions where Cercospora zeae-maydis is prevalent, and thus it is not known if the above methods are effective against Cercospora zeina responsible for GLS in Africa, and other parts of the world such as China. U.S. Pat. No. 5,574,210 is based on RFLP technology which is out-dated and not commonly used in commercial maize breeding programmes.

There is a need for commercially acceptable hybrid and inbred lines displaying a relatively high level of resistance to GLS associated with Cercospora zeina. Thus, methods for identifying maize plants with resistance to GLS with which the aforesaid disadvantages could be overcome or at least minimised are of interest. Also of interest are molecular genetic markers for screening maize plants displaying varying levels of resistance to GLS.

### SUMMARY

The mapping of genetic loci significantly correlated with resistance to gray leaf spot and the application of this knowledge to plant breeding are presented herein. Compositions and methods for identifying maize plants with newly conferred or enhanced resistance to gray leaf spot are provided. Methods of making maize plants that have newly conferred or enhanced resistance to gray leaf spot through marker assisted breeding are also provided, as are plants produced by such methods.

Methods for identifying maize plants that display newly conferred or enhanced resistance to gray leaf spot caused by Cercospora spp. are provided in which at least one allele of a marker locus is detected in the DNA of a maize plant, wherein said marker locus is located within QTL4A, QTL9A, QTL9B, or QTL9C and the allele of the marker locus is associated with the newly conferred or enhanced resistance to gray leaf spot, and a maize plant is selected if it has an allele associated with newly conferred or enhanced resistance to gray leaf spot.

The marker locus may be located within QTL4A, which can be defined by and includes markers bnlg1927 and CPGR.00012, and the allele of the marker locus may be associated with one or more of the following: a product of 192 bp in size when amplified with primers having SEQ ID NOs:29 and 30; an “A” at ZM_C4_183209964; a “C” at ZM_C4_183640675; a “C” at ZM_C4_189294989; a “C” at ZM_C4_187988553; a “C” at CPGR.00012; a “C” at CPGR.00015; an “A” at CPGR.00086; a “T” at CPGR.00090; a “C” at CPGR.00016; a “G” at CPGR.00038; a “G” at CPGR.00098; a product of 123 bp in size when amplified with primers having SEQ ID NOs:31 and 32; and a “G” at CPGR.00102.

The marker locus may be located within QTL9A, which can be defined by and includes markers ZM_C9_124028957 and ZM_C9_131517485, and the allele of the marker locus may be associated with one or more of the following: a “C” at ZM_C9_124028957; a “T” at ZM_C9_125171993; a “T” at ZM_C9_125804907; a “G” at ZM_C9_126185898; an “A” at ZM_C9_126400936; a “T” at ZM_C9_126401198; a “C” at ZM_C9_127295062; a “C” at ZM_C9_131381146; a “T” at ZM_C9_131517485; a “G” at ZM_C9_130093144; an “A” at ZM_C9_128412180; a “C” at ZM_C9_131161648; and a “G” at ZM_C9_129403817.

The marker locus may be located within QTL9B, which can be defined by and includes markers CPGR.00127 and CPGR.00054, and the allele of the marker locus may be associated with one or more of the following: a “G” at ZM_C9_139961409; a “C” at ZM_C9_142658967; a “C” at CPGR.00053; an “A” at CPGR.00125; a “T” at CPGR.00054; a “C” at CPGR.00127; an “A” at CPGR.00131; an “A” at CPGR.00120; a product of 216 bp in size when amplified with primers having SEQ ID NOs:35 and 36; and a product of 78 bp in size when amplified with primers having SEQ ID NOs:33 and 34.

The marker locus may be located within QTL9C, which can be defined by and includes markers umc1675 and ZM_C9_152795210, and the allele of the marker locus may be associated with any of the following: a “G” at ZM_C9_151296063; a “C” at ZM_C9_151687245; a “T” at ZM_C9_152795210; and a product of 155 bp in size when amplified with primers having SEQ ID NOs:37 and 38.

In another embodiment, a method of introgressing a QTL allele associated with newly conferred or enhanced resistance to gray leaf spot caused by Cercospora spp. into a maize plant is provided. Such method includes: crossing a first maize plant comprising a QTL allele associated with newly conferred or enhanced resistance to gray leaf spot with a second maize plant to obtain a population of progeny plants; and screening the progeny plants with at least one marker located within 10 cM of any of the following: ZM_C4_183209964; ZM_C4_183640675; ZM_C4_189294989; ZM_C4_187988553; ZM_C9_124028957; ZM_C9_125171993; ZM_C9_125804907; ZM_C9_126185898; ZM_C9_126400936; ZM_C9_126401198; ZM_C9_127295062; ZM_C9_131381146; ZM_C9_131517485; ZM_C9_130093144; ZM_C9_128412180; ZM_C9_131161648; ZM_C9_129403817; ZM_C9_139961409; ZM_C9_142658967; ZM_C9_151296063; ZM_C9_151687245; ZM_C9_152795210; CPGR.00012; CPGR.00015; CPGR.00086; CPGR.00090; CPGR.00016; CPGR.00038; CPGR.00098; CPGR.00102; CPGR.00053; CPGR.00125; CPGR.00054; CPGR.00127; CPGR.00131; CPGR.00120; bnlg1927; mmc0321; umc1733; bnlg1191; and umc1675; where the marker comprises an allele associated with newly conferred or enhanced resistance to gray leaf spot; and determining if the progeny plants comprise the QTL allele associated with newly conferred or enhanced resistance to gray leaf spot.

In another embodiment, a method of identifying a maize plant containing at least one allele of a marker locus associated with newly conferred or enhanced resistance to gray leaf spot caused by Cercospora spp. is provided in which a maize plant is genotyped with at least one marker that is linked to any of the following: ZM_C4_183209964; ZM_C4_183640675; ZM_C4_189294989; ZM_C4_187988553; ZM_C9_124028957; ZM_C9_125171993; ZM_C9_125804907; ZM_C9_126185898; ZM_C9_126400936; ZM_C9_126401198; ZM_C9_127295062; ZM_C9_131381146; ZM_C9_131517485; ZM_C9_130093144; ZM_C9_128412180; ZM_C9_131161648; ZM_C9_129403817; ZM_C9_139961409; ZM_C9_142658967; ZM_C9_151296063; ZM_C9_151687245; ZM_C9_152795210; CPGR.00012; CPGR.00015; CPGR.00086; CPGR.00090; CPGR.00016; CPGR.00038; CPGR.00098; CPGR.00102; CPGR.00053; CPGR.00125; CPGR.00054; CPGR.00127; CPGR.00131; CPGR.00120; bnlg1927; mmc0321; umc1733; bnlg1191; and umc1675; and a maize plant containing at least one allele at the marker that is associated with newly conferred or enhanced resistance to gray leaf spot is selected.

The marker locus may be linked to any of the following: ZM_C4_183209964; ZM_C4_183640675; ZM_C4_189294989; ZM_C4_187988553; ZM_C9_124028957; ZM_C9_125171993; ZM_C9_125804907; ZM_C9_126185898; ZM_C9_126400936; ZM_C9_126401198; ZM_C9_127295062; ZM_C9_131381146; ZM_C9_131517485; ZM_C9_130093144; ZM_C9_128412180; ZM_C9_131161648; ZM_C9_129403817; ZM_C9_139961409; ZM_C9_142658967; ZM_C9_151296063; ZM_C9_151687245; ZM_C9_152795210; CPGR.00012; CPGR.00015; CPGR.00086; CPGR.00090; CPGR.00016; CPGR.00038; CPGR.00098; CPGR.00102; CPGR.00053; CPGR.00125; CPGR.00054; CPGR.00127; CPGR.00131; CPGR.00120; bnlg1927; mmc0321; umc1733; bnlg1191; and umc1675; by 10 cM, 9 cM, 8, cM, 7 cM, 6 cM, 5 cM, 4 cM, 3 cM, 2 cM, 1 cM, 0.9 cM, 0.8 cM, 0.7 cM, 0.6 cM, 0.5 cM, 0.4 cM, 0.3 cM, 0.2 cM, 0.1 cM or less on a single meiosis based genetic map.

In another embodiment, a method of identifying and/or selecting a maize plant with newly conferred or enhanced resistance to gray leaf spot caused by Cercospora spp. in which the method includes: detecting in a maize plant one or more marker alleles that are linked to and associated with a haplotype comprising:
i. a haplotype comprising: a “C” at ZM_C4_183640675; a “C” at ZM_C4_187988553; and a “C” at ZM_C4_189294989;
ii. a haplotype comprising: a “T” at ZM_C9_125804907; a “G” at ZM_C9_26185898; an “A” at ZM_C9_126400936; and a “T” at ZM_C9_126401198;
iii. a haplotype comprising: a “G” at ZM_C9_139961409 and a “C” at ZM_C9_142658967; or
iv. a haplotype comprising: a “G” at ZM_C9_151296063; a “C” at ZM_C9_151687245; and a “T” at ZM_C9_152795210; and
   selecting a maize plant having the one or more marker alleles. The one or more marker alleles may be linked to either haplotype by 10 cM, 9 cM, 8, cM, 7 cM, 6 cM, 5 cM, 4 cM, 3 cM, 2 cM, 1 cM, 0.9 cM, 0.8 cM, 0.7 cM, 0.6 cM, 0.5 cM, 0.4 cM, 0.3 cM, 0.2 cM, 0.1 cM or less on a single meiosis based genetic map.

In any of the methods above, the gray leaf spot may be caused by Cercospora zeina.

Also provided are plants generated by any of the methods presented herein.

### BRIEF DESCRIPTION OF THE DRAWINGS AND SEQUENCE LISTING

The invention can be more fully understood from the following detailed description and the accompanying Drawings and Sequence Listing which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984), which are herein incorporated by reference in their entirety. The symbols and format used for nucleotide and amino acid sequence data comply with the rules set forth in 37 C.F.R. §1.822.

FIG. 1 is a field evaluation of resistance to GLS in maize plants from the RIL population derived from the cross between XR411 and JS891. The y-axis shows the number of RILs with a particular GLS disease score and the x-axis shows the GLS disease severity score on a scale of 1-9. There were no RILs with a score of 1. Higher scores represent higher GLS disease.

SEQ ID NO:1 is the reference sequence of marker ZM_C4_183209964.

SEQ ID NO:2 is the reference sequence of marker ZM_C4_183640675.

SEQ ID NO:3 is the reference sequence of marker ZM_C4_189294989.

SEQ ID NO:4 is the reference sequence of marker ZM_C4_187988553.

SEQ ID NO:5 is the reference sequence of marker ZM_C9_124028957.

SEQ ID NO:6 is the reference sequence of marker ZM_C9_125171993.

SEQ ID NO:7 is the reference sequence of marker ZM_C9_125804907.

SEQ ID NO:8 is the reference sequence of marker ZM_C9_126185898.

SEQ ID NO:9 is the reference sequence of marker ZM_C9_126400936.

SEQ ID NO:10 is the reference sequence of marker ZM_C9_126401198.

SEQ ID NO:11 is the reference sequence of marker ZM_C9_139961409.

SEQ ID NO:12 is the reference sequence of marker ZM_C9_142658967.

SEQ ID NO:13 is the reference sequence of marker ZM_C9_151296063.

SEQ ID NO:14 is the reference sequence of marker ZM_C9_151687245.

SEQ ID NO:15 is the reference sequence of marker CPGR.00012.

SEQ ID NO:16 is the reference sequence of marker CPGR.00015.

SEQ ID NO:17 is the reference sequence of marker CPGR.00086.

SEQ ID NO:18 is the reference sequence of marker CPGR.00090.

SEQ ID NO:19 is the reference sequence of marker CPGR.00016.

SEQ ID NO:20 is the reference sequence of marker CPGR.00038.

SEQ ID NO:21 is the reference sequence of marker CPGR.00098.

SEQ ID NO:22 is the reference sequence of marker CPGR.00102.

SEQ ID NO:23 is the reference sequence of marker CPGR.00053.

SEQ ID NO:24 is the reference sequence of marker CPGR.00125.

SEQ ID NO:25 is the reference sequence of marker CPGR.00054.

SEQ ID NO:26 is the reference sequence of marker CPGR.00127.

SEQ ID NO:27 is the reference sequence of marker CPGR.00131.

SEQ ID NO:28 is the reference sequence of marker CPGR.00120.

SEQ ID NO:29 is the sequence of the bnlg1927 forward primer.

SEQ ID NO:30 is the sequence of the bnlg1927 reverse primer.

SEQ ID NO:31 is the sequence of the mmc0321 forward primer.

SEQ ID NO:32 is the sequence of the mmc0321 reverse primer.

SEQ ID NO:33 is the sequence of the umc1733 forward primer.

SEQ ID NO:34 is the sequence of the umc1733 reverse primer.

SEQ ID NO:35 is the sequence of the bnlg1191 forward primer.

SEQ ID NO:36 is the sequence of the bnlg1191 reverse primer.

SEQ ID NO:37 is the sequence of the umc1675 forward primer.

SEQ ID NO:38 is the sequence of the umc1675 reverse primer.

SEQ ID NO:39 is the reference sequence of marker ZM_C9_127295062.

SEQ ID NO:40 is the reference sequence of marker ZM_C9_131381146.

SEQ ID NO:41 is the reference sequence of marker ZM_C9_131517485.

SEQ ID NO:42 is the reference sequence of marker ZM_C9_130093144.

SEQ ID NO:43 is the reference sequence of marker ZM_C9_128412180.

SEQ ID NO:44 is the reference sequence of marker ZM_C9_131161648.

SEQ ID NO:45 is the reference sequence of marker ZM_C9_129403817.

SEQ ID NO:46 is the reference sequence of marker ZM_C9_152795210.

### DETAILED DESCRIPTION

Maize marker loci that demonstrate statistically significant co-segregation with the gray leaf spot resistance trait are provided herein. Detection of these loci or additional linked loci can be used in marker assisted selection as part of a maize breeding program to produce maize plants that have resistance to gray leaf spot.

The following definitions are provided as an aid to understand the present disclosure.

It is to be understood that the disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Unless otherwise indicated, nucleic acids are written left to right in 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the disclosure pertains. Although any methods and materials similar or equivalent to those described herein can be used for testing of the subject matter recited in the current disclosure, the preferred materials and methods are described herein. In describing and claiming the subject matter of the current disclosure, the following terminology will be used in accordance with the definitions set out below.

The term “allele” refers to one of two or more different nucleotide sequences that occur at a specific locus.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An “amplicon” is an amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An allele is “associated with” a trait when it is part of or linked to a DNA sequence or allele that affects the expression of the trait. The presence of the allele is an indicator of how the trait will be expressed.

“Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F₁ generation; the term “BC₁” then refers to the second use of the recurrent parent, “BC₂” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

A “chromosome” is a single piece of coiled DNA containing many genes that act and move as a unity during cell division and therefore can be said to be linked. It can also be referred to as a “linkage group”.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “complement” refers to a nucleotide sequence that is complementary to a given nucleotide sequence, i.e. the sequences are related by the Watson-Crick base-pairing rules.

The term “contiguous DNA” refers to an uninterrupted stretch of genomic DNA represented by partially overlapping pieces or contigs.

When referring to the relationship between two genetic elements, such as a genetic element contributing to gray leaf spot resistance and a proximal marker, “coupling” phase linkage indicates the state where the “favorable” allele at the gray leaf spot resistance locus is physically associated on the same chromosome strand as the “favorable” allele of the respective linked marker locus. In coupling phase, both favorable alleles are inherited together by progeny that inherit that chromosome strand.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

A plant referred to herein as “diploid” has two sets (genomes) of chromosomes.

A plant referred to herein as a “doubled haploid” is developed by doubling the haploid set of chromosomes (i.e., half the normal number of chromosomes). A doubled haploid plant has two identical sets of chromosomes, and all loci are considered homozygous.

An “elite line” is any line that has resulted from breeding and selection for superior agronomic performance.

An “exotic maize strain” or an “exotic maize germplasm” is a strain derived from a maize plant not belonging to an available elite maize line or strain of germ plasm. In the context of a cross between two maize plants or strains of germ plasm, an exotic germ plasm is not closely related by descent to the elite germ plasm with which it is crossed. Most commonly, the exotic germplasm is not derived from any known elite line of maize, but rather is selected to introduce novel genetic elements (typically novel alleles) into a breeding program.

A “favorable allele” is the allele at a particular locus (a marker, a QTL, etc.) that confers, or contributes to, an agronomically desirable phenotype, e.g., newly conferred or enhanced resistance to gray leaf spot, and that allows the identification of plants with that agronomically desirable phenotype. A favorable allele of a marker is a marker allele that segregates with the favorable phenotype.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g. markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci. Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

As used herein, “gray leaf spot resistance” refers to enhanced resistance or tolerance to a fungal pathogen that causes gray leaf spot when compared to a control plant. Effects may vary from a slight increase in tolerance to the effects of the fungal pathogen (e.g., partial inhibition) to total resistance such that the plant is unaffected by the presence of the fungal pathogen. An increased level of resistance against a particular fungal pathogen or against a wider spectrum of fungal pathogens constitutes “enhanced” or improved fungal resistance. The embodiments of the disclosure will enhance or improve resistance to the fungal pathogen that causes gray leaf spot, such that the resistance of the plant to a fungal pathogen or pathogens will increase. The term “enhance” refers to improve, increase, amplify, multiply, elevate, raise, and the like. Thus, plants described herein as being resistant to gray leaf spot can also be described as being resistant to infection by Cercospora spp. or having ‘enhanced resistance’ to infection by Cercospora spp. Members of the Cercospora spp. include Cercospora zeae-maydis and Cercospora zeina.

A plant referred to as “haploid” has a single set (genome) of chromosomes.

A “haplotype” is the genotype of an individual at a plurality of genetic loci, i.e. a combination of alleles. Typically, the genetic loci described by a haplotype are physically and genetically linked, i.e., on the same chromosome segment.

The term “heterogeneity” is used to indicate that individuals within the group differ in genotype at one or more specific loci.

The heterotic response of material, or “heterosis”, can be defined by performance which exceeds the average of the parents (or high parent) when crossed to other dissimilar or unrelated groups.

An individual is “heterozygous” if more than one allele type is present at a given locus (e.g., a diploid individual with one copy each of two different alleles).

The term “homogeneity” indicates that members of a group have the same genotype at one or more specific loci.

An individual is “homozygous” if the individual has only one type of allele at a given locus (e.g., a diploid individual has a copy of the same allele at a locus for each of two homologous chromosomes).

The term “hybrid” refers to the progeny obtained between the crossing of at least two genetically dissimilar parents.

“Hybridization” or “nucleic acid hybridization” refers to the pairing of complementary RNA and DNA strands as well as the pairing of complementary DNA single strands.

The term “hybridize” means to form base pairs between complementary regions of nucleic acid strands.

An “IBM genetic map” can refer to any of following maps: IBM, IBM2, IBM2 neighbors, IBM2 FPCO507, IBM2 2004 neighbors, IBM2 2005 neighbors, IBM2 2005 neighbors frame, IBM2 2008 neighbors, IBM2 2008 neighbors frame, or the latest version on the maizeGDB website. IBM genetic maps are based on a B73×Mo17 population in which the progeny from the initial cross were random-mated for multiple generations prior to constructing recombinant inbred lines for mapping. Newer versions reflect the addition of genetic and BAC mapped loci as well as enhanced map refinement due to the incorporation of information obtained from other genetic maps or physical maps, cleaned date, or the use of new algorithms.

The term “inbred” refers to a line that has been bred for genetic homogeneity.

The term “indel” refers to an insertion or deletion, wherein one line may be referred to as having an inserted nucleotide or piece of DNA relative to a second line, or the second line may be referred to as having a deleted nucleotide or piece of DNA relative to the first line.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F₂; the IBM2 maps consist of multiple meioses). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r², which is calculated using the formula described by Hill, W. G. and Robertson, A, Theor. Appl. Genet. 38:226-231(1968). When r²=1, complete LD exists between the two marker loci, meaning that the markers have not been separated by recombination and have the same allele frequency. The r² value will be dependent on the population used. Values for r² above ⅓ indicate sufficiently strong LD to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome, e.g. where a nucleotide, gene, sequence, or marker is located.

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

“Maize” refers to a plant of the Zea mays L. ssp. mays and is also known as “corn”.

The term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (of MAS) is a process by which individual plants are selected based on marker genotypes.

“Marker assisted counter-selection” is a process by which marker genotypes are used to identify plants that will not be selected, allowing them to be removed from a breeding program or planting.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

An allele “negatively” correlates with a trait when it is linked to it and when presence of the allele is an indicator that a desired trait or trait form will not occur in a plant comprising the allele.

“Nucleotide sequence”, “polynucleotide”, “nucleic acid sequence”, and “nucleic acid fragment” are used interchangeably and refer to a polymer of RNA or

DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “phenotype”, “phenotypic trait”, or “trait” can refer to the observable expression of a gene or series of genes. The phenotype can be observable to the naked eye, or by any other means of evaluation known in the art, e.g., weighing, counting, measuring (length, width, angles, etc.), microscopy, biochemical analysis, or an electromechanical assay. In some cases, a phenotype is directly controlled by a single gene or genetic locus, i.e., a “single gene trait” or a “simply inherited trait”. In the absence of large levels of environmental variation, single gene traits can segregate in a population to give a “qualitative” or “discrete” distribution, i.e. the phenotype falls into discrete classes. In other cases, a phenotype is the result of several genes and can be considered a “multigenic trait” or a “complex trait”. Multigenic traits segregate in a population to give a “quantitative” or “continuous” distribution, i.e. the phenotype cannot be separated into discrete classes. Both single gene and multigenic traits can be affected by the environment in which they are being expressed, but multigenic traits tend to have a larger environmental component.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

An allele “positively” correlates with a trait when it is linked to it and when presence of the allele is an indicator that the desired trait or trait form will occur in a plant comprising the allele.

The “probability value” or “p-value” is the statistical likelihood that the particular combination of a phenotype and the presence or absence of a particular marker allele is random. Thus, the lower the probability score, the greater the likelihood that a locus and a phenotype are associated. The probability score can be affected by the proximity of the first locus (usually a marker locus) and the locus affecting the phenotype, plus the magnitude of the phenotypic effect (the change in phenotype caused by an allele substitution). In some aspects, the probability score is considered “significant” or “nonsignificant”. In some embodiments, a probability score of 0.05 (p=0.05, or a 5% probability) of random assortment is considered a significant indication of association. However, an acceptable probability can be any probability of less than 50% (p=0.5). For example, a significant probability can be less than 0.25, less than 0.20, less than 0.15, less than 0.1, less than 0.05, less than 0.01, or less than 0.001.

The term “progeny” refers to the offspring generated from a cross.

A “progeny plant” is a plant generated from a cross between two plants.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question.

“Recombinant inbred lines” or RILs are the product of an initial cross between two parent lines and the subsequent selfing to produce homozygous lines.

A “reference sequence” or a “consensus sequence” is a defined sequence used as a basis for sequence comparison. The reference sequence for a PHM marker is obtained by sequencing a number of lines at the locus, aligning the nucleotide sequences in a sequence alignment program (e.g. Sequencher), and then obtaining the most common nucleotide sequence of the alignment. Polymorphisms found among the individual sequences are annotated within the consensus sequence. A reference sequence is not usually an exact copy of any individual DNA sequence, but represents an amalgam of available sequences and is useful for designing primers and probes to polymorphisms within the sequence.

In “repulsion” phase linkage, the “favorable” allele at the locus of interest is physically linked with an “unfavorable” allele at the proximal marker locus, and the two “favorable” alleles are not inherited together (i.e., the two loci are “out of phase” with each other).

A “topeross test” is a test performed by crossing each individual (e.g. a selection, inbred line, clone or progeny individual) with the same pollen parent or “tester”, usually a homozygous line.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

An “unfavorable allele” of a marker is a marker allele that segregates with the unfavorable plant phenotype, therefore providing the benefit of identifying plants that can be removed from a breeding program or planting.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics”, “agronomic traits”, and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the CLUSTAL V method of alignment (Higgins and Sharp, CABIOS. 5:151 153 (1989)) with the default parameters (GAP PENALTY=10, GAP LENGTH PENALTY=10). Default parameters for pairwise alignments and calculation of percent identity of protein sequences using the CLUSTAL V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the CLUSTAL V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### Genetic Mapping

It has been recognized for quite some time that specific genetic loci correlating with particular phenotypes, such as resistance to gray leaf spot, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as the gray leaf spot resistance trait. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) Population-based association analysis (i.e. association mapping) and 2) Traditional linkage analysis.

### Association Mapping

Understanding the extent and patterns of linkage disequilibrium (LD) in the genome is a prerequisite for developing efficient association approaches to identify and map quantitative trait loci (QTL). Linkage disequilibrium (LD) refers to the non-random association of alleles in a collection of individuals. When LD is observed among alleles at linked loci, it is measured as LD decay across a specific region of a chromosome. The extent of the LD is a reflection of the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al., (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Elite breeding lines provide a valuable starting point for association analyses. Association analyses use quantitative phenotypic scores (e.g., disease tolerance rated from one to nine for each maize line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable maize map (for example, a composite map) can optionally be used to help observe distribution of the identified QTL markers and/or QTL marker clustering using previously determined map locations of the markers.

### Traditional Linkage Analysis

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Maize marker loci that demonstrate statistically significant co-segregation with the gray leaf spot resistance trait, as determined by traditional linkage analysis, are provided herein. Detection of these loci or additional linked loci can be used in marker assisted maize breeding programs to produce plants having resistance to gray leaf spot (whether that resistance is newly conferred or enhanced).

Activities in marker assisted maize breeding programs may include but are not limited to: selecting among new breeding populations to identify which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germ plasm improvement activities based on presence of favorable nucleic acid sequences.

### QTL Locations

QTLs on maize chromosomes 4 and 9 were identified as being associated with the gray leaf spot resistance trait using traditional linkage mapping analysis (Example 5). QTL4A was found to be delimited by markers bnlg1927 and CPGR.00012; QTL9A was found to be delimited by markers ZM_C9_124028957 and ZM_C9_131517485; QTL9B was found to be delimited by markers CPGR.00127 and CPGR.00054; and QTL9C was found to be delimited by markers umc1675 and ZM_C9_152795210 (Table 5).

### Chromosomal Intervals

Chromosomal intervals that correlate with the gray leaf spot resistance trait are provided. A variety of methods well known in the art are available for identifying chromosomal intervals. The boundaries of such chromosomal intervals are drawn to encompass markers that will be linked to the gene(s) controlling the trait of interest. In other words, the chromosomal interval is drawn such that any marker that lies within that interval (including the terminal markers that define the boundaries of the interval) can be used as a marker for the gray leaf spot resistance trait. Tables 2, 3, and 5 identify markers within the QTL regions QTL4A, QTL9A, QTL9B, and QTL9C that were shown herein to associate with the gray leaf spot resistance trait and that are linked to a gene(s) controlling gray leaf spot resistance. Reference sequences for each of the markers are represented by SEQ ID NOs:1-28 and 39-46.

Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identify the same QTL or two different QTL. Regardless, knowledge of how many QTL are in a particular interval is not necessary to make or practice that which is presented in the current disclosure.

The QTL4A interval may encompass any of the markers identified herein as being associated with the gray leaf spot resistance trait including: ZM_C4_183209964; ZM_C4_183640675; ZM_C4_189294989; ZM_C4_187988553; CPGR.00012; CPGR.00015; CPGR.00086; CPGR.00090; CPGR.00016; CPGR.00038; CPGR.00098; CPGR.00102; bnlg1927; and mmc0321. The QTL4A interval, for example, may be defined by markers bnlg1927 and CPGR.00012 (Table 5), which are separated by the greatest distance on the physical map. Any marker located within these intervals can find use as a marker for gray leaf spot resistance and can be used in the context of the methods presented herein to identify and/or select maize plants that have resistance to gray leaf spot, whether it is newly conferred or enhanced compared to a control plant.

The QTL9A interval may encompass any of the markers identified herein as being associated with the gray leaf spot resistance trait including: ZM_C9_124028957; ZM_C9_125171993; ZM_C9_125804907; ZM_C9_126185898; ZM_C9_126400936; ZM_C9_126401198; ZM_C9_127295062; ZM_C9_128412180; ZM_C9_129403817; ZM_C9_130093144; ZM_C9_131161648; ZM_C9_131381146; and ZM_C9_131517485. The QTL9A interval, for example, may be defined by markers ZM_C9_124028957 and ZM_C9_131517485 (Table 5), which are separated by the greatest distance on the physical map. Any marker located within these intervals can find use as a marker for gray leaf spot resistance and can be used in the context of the methods presented herein to identify and/or select maize plants that have resistance to gray leaf spot, whether it is newly conferred or enhanced compared to a control plant.

The QTL9B interval may encompass any of the markers identified herein as being associated with the gray leaf spot resistance trait including: ZM_C9_139961409; ZM_C9_142658967; CPGR.00053; CPGR.00125; CPGR.00054; CPGR.00127; CPGR.00131; CPGR.00120; umc1733; and bnlg1191. The QTL9B interval, for example, may be defined by markers CPGR.00127 and CPGR.00054 (Table 5), which are separated by the greatest distance on the physical map. Any marker located within these intervals can find use as a marker for gray leaf spot resistance and can be used in the context of the methods presented herein to identify and/or select maize plants that have resistance to gray leaf spot, whether it is newly conferred or enhanced compared to a control plant.

The QTL9C interval may encompass any of the markers identified herein as being associated with the gray leaf spot resistance trait including: ZM_C9_151296063; ZM_C9_151687245; ZM_C9_152795210; and umc1675. The QTL9C interval, for example, may be defined by markers umc1675 and ZM_C9_152795210 (Table 5), which are separated by the greatest distance on the physical map. Any marker located within these intervals can find use as a marker for gray leaf spot resistance and can be used in the context of the methods presented herein to identify and/or select maize plants that have resistance to gray leaf spot, whether it is newly conferred or enhanced compared to a control plant.

Chromosomal intervals can also be defined by markers that are linked to (show linkage disequilibrium with) a QTL marker, and r² is a common measure of linkage disequilibrium (LD) in the context of association studies. If the r² value of LD between a marker locus disclosed herein, for example, and another marker locus in close proximity (i.e. “linked”) is greater than ⅓ (Ardlie et al., Nature Reviews Genetics 3:299-309 (2002)), the loci are in linkage disequilibrium with one another.

### Markers and Linkage Relationships

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with the gray leaf spot resistance trait, it is important to note that the marker locus is not necessarily responsible for the expression of the gray leaf spot resistant phenotype. For example, it is not a requirement that the marker polynucleotide sequence be part of a gene that is responsible for the gray leaf spot resistant phenotype (for example, is part of the gene open reading frame). The association between a specific marker allele and the gray leaf spot resistance trait is due to the original “coupling” linkage phase between the marker allele and the allele in the ancestral maize line from which the allele originated. Eventually, with repeated recombination, crossing over events between the marker and genetic locus can change this orientation. For this reason, the favorable marker allele may change depending on the linkage phase that exists within the parent having resistance to gray leaf spot that is used to create segregating populations. This does not change the fact that the marker can be used to monitor segregation of the phenotype. It only changes which marker allele is considered favorable in a given segregating population.

Methods presented herein include detecting the presence of one or more marker alleles associated with gray leaf spot resistance in a maize plant and then identifying and/or selecting maize plants that have favorable alleles at those marker loci. Markers listed in Tables 2, 3, and 5 have been identified herein as being associated with the gray leaf spot resistance trait and hence can be used to predict gray leaf spot resistance in a maize plant. Any marker within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM, 0.75 cM, 0.5 cM or 0.25 cM (based on a single meiosis based genetic map) of any of the markers in Tables 2, 3, and 5 could also be used to predict gray leaf spot resistance in a maize plant.

### Marker Assisted Selection

Molecular markers can be used in a variety of plant breeding applications (e.g. see Staub et al. (1996) Hortscience 31: 729-741; Tanksley (1983) Plant Molecular Biology Reporter. 1: 3-8). One of the main areas of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of integrated linkage maps of the maize genome containing increasing densities of public maize markers has facilitated maize genetic mapping and MAS. See, e.g. the IBM2 Neighbors maps, which are available online on the MaizeGDB website.

The key components to the implementation of MAS are: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6) Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396). SSRs are highly suited to mapping and MAS as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment. An SSR service for maize is available to the public on a contractual basis by DNA Landmarks in Saint-Jean-sur-Richelieu, Quebec, Canada.

Various types of fragment length polymorphism (FLP) markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele ‘T’ for a specific line or variety with gray leaf spot resistance, but the allele ‘T’ might also occur in the maize breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

In addition to SSRs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the maize species, or even across other species that have been genetically or physically aligned with maize, such as rice, wheat, barley or sorghum.

In general, MAS uses polymorphic markers that have been identified as having a significant likelihood of co-segregation with a trait such as the gray leaf spot resistance trait. Such markers are presumed to map near a gene or genes that give the plant its gray leaf spot resistant phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with gray leaf spot resistance can be selected for by detecting one or more marker alleles, and in addition, progeny plants derived from those plants can also be selected. Hence, a plant containing a desired genotype in a given chromosomal region (i.e. a genotype associated with gray leaf spot resistance) is obtained and then crossed to another plant. The progeny of such a cross would then be evaluated genotypically using one or more markers and the progeny plants with the same genotype in a given chromosomal region would then be selected as having gray leaf spot resistance.

Markers were identified from linkage mapping as being associated with the gray leaf spot resistance trait. The SSR markers associated with the gray leaf spot resistance trait are found in Table 3 and are public markers. The primer sequences for the SSR markers are represented by SEQ ID NOs:29-38. The SNP markers associated with the gray leaf spot resistance trait are provided in Table 2. Reference sequences for the SNP markers are represented by SEQ ID NOs:1-28 and 39-46. SNP positions are identified within the marker reference sequences (Table 2).

Markers could be used alone or in combination either to select for favorable QTL alleles associated with newly conferred or enhanced resistance to gray leaf spot or to counter-select unfavorable QTL alleles associated with gray leaf spot susceptibility. Marker alleles identified in Tables 2 and 3 as co-segregating with GLS resistance can be used to identify and select maize plants with newly conferred or enhanced resistance to gray leaf spot. Alternatively, marker alleles identified in Tables 2 and 3 as co-segregating with GLS susceptibility can be used to identify and counter select GLS susceptible plants. For instance, in the latter, an allele can be used for exclusionary purposes during breeding to identify alleles that negatively correlate with resistance, in order to eliminate susceptible plants from subsequent rounds of breeding.

SNPs could be used alone or in combination (i.e. a SNP haplotype) to select for favorable QTL alleles associated with gray leaf spot resistance.

For example, a SNP haplotype at QTL4A may comprise: a “C” at ZM_C4_183640675; a “C” at ZM_C4_187988553; and a “C” at ZM_C4_189294989. A SNP haplotype at QTL9A may comprise: a “T” at ZM_C4125804907; a “G” at ZM_C9_126185898; an “A” at ZM_C9_126400936; and a “T” at ZM_C9_126401198. A SNP haplotype at QTL9B may comprise: a “G” at ZM_C9_139961409 and a “C” at ZM_C9_42658967. A SNP haplotype at QTL9C may comprise: a “G” at ZM_C9_151296063; a “C” at ZM_C9_151687245; and a “T” at ZM_C9_152795210.

The skilled artisan would expect that there might be additional polymorphic sites at marker loci in and around the markers identified herein, wherein one or more polymorphic sites is in linkage disequilibrium (LD) with an allele at one or more of the polymorphic sites in the haplotype and thus could be used in a marker assisted selection program to introgress a QTL allele of interest. Two particular alleles at different polymorphic sites are said to be in LD if the presence of the allele at one of the sites tends to predict the presence of the allele at the other site on the same chromosome (Stevens, Mol. Diag. 4:309-17 (1999)). The marker loci can be located within 10 cM, 5 cM, 2 cM, or 1 cM (on a single meiosis based genetic map) of the gray leaf spot resistance trait QTL.

The skilled artisan would understand that allelic frequency (and hence, haplotype frequency) can differ from one germ plasm pool to another. Germ plasm pools vary due to maturity differences, heterotic groupings, geographical distribution, etc. As a result, SNPs and other polymorphisms may not be informative in some germplasm pools.

### Plant Compositions

Maize plants identified and/or selected by any of the methods described above are also of interest.

### Seed Treatments

To protect and to enhance yield production and trait technologies, seed treatment options can provide additional crop plan flexibility and cost effective control against insects, weeds and diseases, thereby further enhancing the subject matter described herein. Seed material can be treated, typically surface treated, with a composition comprising combinations of chemical or biological herbicides, herbicide safeners, insecticides, fungicides, germination inhibitors and enhancers, nutrients, plant growth regulators and activators, bactericides, nematicides, avicides and/or molluscicides. These compounds are typically formulated together with further carriers, surfactants or application-promoting adjuvants customarily employed in the art of formulation. The coatings may be applied by impregnating propagation material with a liquid formulation or by coating with a combined wet or dry formulation. Examples of the various types of compounds that may be used as seed treatments are provided in The Pesticide Manual: A World Compendium, C.D.S. Tomlin Ed., Published by the British Crop Production Council, which is hereby incorporated by reference.

Some seed treatments that may be used on crop seed include, but are not limited to, one or more of abscisic acid, acibenzolar-S-methyl, avermectin, amitrol, azaconazole, azospirillum, azadirachtin, azoxystrobin, bacillus spp. (including one or more of cereus, firmus, megaterium, pumilis, sphaericus, subtilis and/or thuringiensis), bradyrhizobium spp. (including one or more of betae, canariense, elkanii, iriomotense, japonicum, liaonigense, pachyrhizi and/or yuanmingense), captan, carboxin, chitosan, clothianidin, copper, cyazypyr, difenoconazole, etidiazole, fipronil, fludioxonil, fluquinconazole, flurazole, fluxofenim, harpin protein, imazalil, imidacloprid, ipconazole, isoflavenoids, lipo-chitooligosaccharide, mancozeb, manganese, maneb, mefenoxam, metalaxyl, metconazole, PCNB, penflufen, penicillium, penthiopyrad, permethrine, picoxystrobin, prothioconazole, pyraclostrobin, rynaxypyr, S-metolachlor, saponin, sedaxane, TCMTB, tebuconazole, thiabendazole, thiamethoxam, thiocarb, thiram, tolclofos-methyl, triadimenol, trichoderma, trifloxystrobin, triticonazole and/or zinc. PCNB seed coat refers to EPA registration number 00293500419, containing quintozen and terrazole. TCMTB refers to 2-(thiocyanomethylthio) benzothiazole.

Seeds that produce plants with specific traits (such as gray leaf spot resistance) may be tested to determine which seed treatment options and application rates may complement such plants in order to enhance yield. For example, a plant with good yield potential but head smut susceptibility may benefit from the use of a seed treatment that provides protection against head smut, a plant with good yield potential but cyst nematode susceptibility may benefit from the use of a seed treatment that provides protection against cyst nematode, and so on. Further, the good root establishment and early emergence that results from the proper use of a seed treatment may result in more efficient nitrogen use, a better ability to withstand drought and an overall increase in yield potential of a plant or plants containing a certain trait when combined with a seed treatment.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Generation and Evaluation of the Segregating Population

Maize line XR411, which has a favorable GLS resistance phenotype, and maize line JS891 were crossed to produce F₁ progeny plants, which were then selfed to produced F₂ progeny. Each F₂ progeny plant was selfed and a recombinant inbred line (RIL) population was produced by the process of single seed descent over at least four additional generations.

### Example 2

### GLS Evaluation of Maize Plants

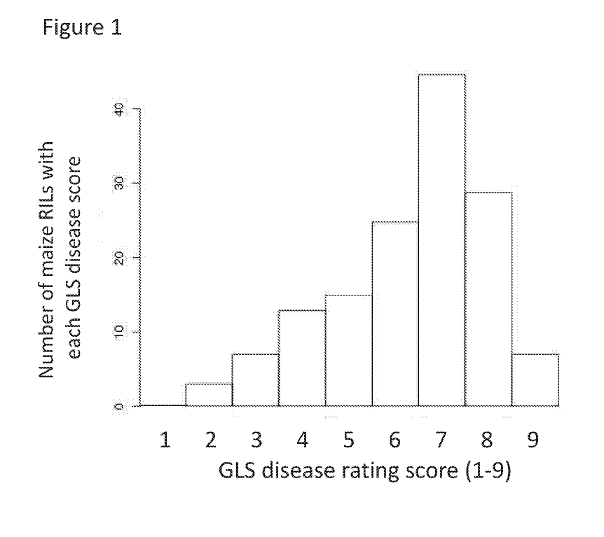

The recombinant inbred line (RIL) population generated in EXAMPLE 1 was evaluated for GLS resistance by means of a numeric score ranging from 1 to 9. The scale was applied as follows: 1=no GLS disease symptoms on leaf samples, 3=GLS lesions on lower leaves and no lesions on leaves above the ear, 5=GLS lesions on most leaves and some lower leaves dead, 7=many GLS lesions on all leaves above the ear and lower leaves dead, and 9=nearly all leaves are dead from coalesced GLS lesions. FIG. 1 shows the field evaluation of the maize RIL population (XR411×JS891) using the whole maize plant 1-9 disease scale to illustrate that there is a range of GLS resistant RILs (LHS, low scores) to susceptible RILs (RHS, high scores).

### Example 3

### Genotyping of Recombinant Inbred Line (RIL)

Leaf samples were collected from each RIL progeny plant, and genomic DNA was extracted using a method well-known in the art.

SNP marker analysis was performed using the Infinium assay with a SNP50 BeadChip to obtain SNP marker data for more than 50,000 SNPs across the maize genome for each individual RIL in the maize population (Ganal et al (2011) PloS One 6 (12) e28334). Data for each RIL for a total of 560 SNP markers was obtained and subsequently used to construct the genetic map.

### Example 4

### Construction of a Genetic Linkage Map

Data obtained from the SNP genetic molecular markers of the recombinant inbred line (RIL) population was used to construct the genetic linkage map with regression mapping using JoinMap (Van Ooijen (2006) JoinMap 4, Software for the calculation of genetic linkage maps in experimental populations. Kyazma B. V., Wageningen, Netherlands). A total of 560 markers was used to construct the genetic linkage map using a method well-known in the art, with most gaps between adjacent markers less than 10 cM (centimorgan) in the genetic linkage map.

### Example 5

### Marker-Trait Association Analysis (QTL Mapping)

Composite Interval Mapping method (CIM) was used to detect a marker locus that is associated with GLS resistance. QTL mapping analysis was used to determine which polymorphic marker demonstrates a statistical likelihood of co-segregation with the resistance phenotype.

QTL for GLS resistance in the recombinant inbred line (RIL) population were identified for each field trial based on the genetic map comprised of 560 markers and applying the Composite Interval Mapping (CIM) utility in Windows QTL Cartographer 2.5_011 (Wang S. et al. 2012. Windows QTL Cartographer 2.5. Department of Statistics, North Carolina State University, Raleigh, N.C.) using the standard model 6 with a window size of 10 cM and a 1 cM walk speed. Both forward and backward regression analysis was performed. The statistical significance LOD (logarithm of odds) score threshold was used to declare the presence of QTLs. LOD score provides a measure of the strength of evidence for the presence of a QTL compared to no segregating QTL on a particular chromosome; therefore larger LOD scores correspond to greater evidence for the presence of a QTL. The LOD score (LOD=−log₁₀(H₀/H₁)) was calculated at each interval for the difference in phenotype and genetic difference at a particular locus between genotypic groups (XR411 (genotype A) or JS891 (genotype B)), were H₀ is the hypothesis that there is no difference between groups (no QTL segregate) and H₁ that there is a difference (QTL segregate). The LOD score threshold was obtained from 1000 permutations at a genome-wide significance level of 5% for each field trial (Doerge R W and Churchill G A. 1996. Genetics 142: 285-294).

The genotype groups are based on whether each 10 cM interval is estimated by the CIM algorithm to be derived from XR411 (genotype A) or JS891 (genotype B). An extreme example of a highly significant QTL would be a 10 cM interval for which all RILs that have the XR411 allele at that interval have low GLS scores (for example, 2-4), and all RILs that have the JS891 allele at that interval have high GLS scores (for example, 5-8). This would indicate a resistance QTL derived from XR411 at this interval position on the genomic DNA. The CIM applies a more sophisticated algorithm to marker-trait association than a “single marker” analysis, since it takes into account the effect of flanking markers and other genomic regions. The position of a QTL is defined by its 1- and 2-LOD support intervals which correspond to 95% and 99% confidence intervals, respectively. Epistatic interactions between QTL were assessed using the Multiple Interval Mapping (MIM) utility in Windows QTL Cartographer as previously described (Balint-Kurti P J et al. 2006. Phytopathology 96:1067-1071).

Four QTLs for GLS resistance were identified from the GLS data from the field trials (Table 1). The QTLs were named based on the chromosome that they mapped to on the genetic map, namely QTL4A, QTL9A, QTL9B and QTL9C.

TABLE 1
QTLs for GLS resistance identified in the XR411 × JS891 RIL population.

| | | | | 1-LOD | 2-LOD | LOD | | | Allele | QTL |
|---|---|---|---|---|---|---|---|---|---|---|
| Trait^{a} | Year | Chr | Peak marker^{b} | interval^{c} | interval^{d} | score^{e} | R2^{f} | Additive^{g} | source^{h} | nameⁱ |
| H_09 | 2 | 4 | ZM_C4_189294989 | 94.3-96.6 | 91.2-97.3 | 3.29 | 8.66 | −0.357 | XR411 | QTL4A |
| U_09 | 2 | 4 | ZM_C4_189294989 | 94.6-97.3 | 94.1-97.3 | 4.99 | 14.58 | −0.535 | XR411 | QTL4A |
| U_08 | 1 | 4 | ZM_C4_189294989 | 95.7-96.9 | 94.8-96.9 | 4.39 | 14.42 | −0.620 | XR411 | QTL4A |
| R_10 | 3 | 9 | ZM_C9_126401198 | 60.0-62.5 | 58.4-62.5 | 3.85 | 14.30 | −0.569 | XR411 | QTL9A |
| H_09 | 2 | 9 | ZM_C9_126401198 | 60.0-63.7 | 59.8-64.5 | 5.97 | 17.68 | −0.521 | XR411 | QTL9A |
| R_08 | 1 | 9 | ZM_C9_126401198 | 59.0-62.0 | 57.4-62.5 | 7.71 | 23.89 | −0.498 | XR411 | QTL9A |
| B_09 | 2 | 9 | ZM_C9_126401198 | 60.0-62.4 | 59.8-64.5 | 5.26 | 20.29 | −0.689 | XR411 | QTL9A |
| C_08 | 1 | 9 | ZM_C9_126401198 | 60.0-62.2 | 58.6-63.6 | 6.39 | 16.83 | −0.714 | XR411 | QTL9A |
| B_09 | 2 | 9 | ZM_C9_142658967 | 80.0-83.9 | 77.6-83.9 | 3.13 | 10.36 | 0.484 | JS891 | QTL9B |
| U_09 | 2 | 9 | ZM_C9_151687245 | 100.2-104.7 | 99.0-106.0 | 4.82 | 14.67 | 0.562 | JS891 | QTL9C |
| Mean_z* | | 9 | ZM_C9_126401198 | 60.0-62.5 | 59.8-62.5 | 4.94 | 16.05 | −0.374 | XR411 | QTL9A |

^{a}Trait (field trial) name.
^{b}Peak marker refers to marker on genetic map that is closest to the QTL peak.
^{c}Range in cM that defines 1-LOD interval of QTL.
^{d}Range in cM that defines 2-LOD interval of QTL.
^{e}Log of odds (LOD) value at position of QTL peak.
^{f}Phenotypic variance explained by the QTL (expressed as percentage).
^{g}Additive effect of QTL. For GLS disease ratings, this is based on the one to nine scale employed. Positive values indicate that the allele for resistance was derived from JS891.
^{h}Parental allele associated with increased GLS resistance.
ⁱQTL name. The QTL name (QTL4A, QTL9A, QTL9B or QTL9C).

### Example 6

### Identification of Additional SNPs in the GLS QTL Regions by RNA Sequencing

RNA sequencing was performed to identify additional SNPs in the GLS QTL regions which may have utility in marker assisted breeding and fine-mapping of the QTL. The two parental lines (or pairs of RILs that showed different parental origins in the QTL genomic regions) were subjected to RNA sequencing using methods known in the art (e.g. Hansey et al. 2012. PLoS One 7(3): e33071). Leaf material from maize plants infected with Cercospora spp. was used for RNA extraction and subsequently, RNA sequencing was performed. “QTL region genes” that are positioned between the flanking markers of the QTL regions were selected using the maize inbred line B73 genome sequence, which is publicly available, and the RNA sequencing reads were mapped to the QTL region genes from each of the parents to identify SNPs that are polymorphic between parents XR411 and JS891. The SNP markers were converted into a Golden Gate 96 SNP assay for high-throughput analysis. All the RILs in the population were analyzed using the 96 SNP assay, and genetic linkage mapping was carried out to determine if the SNPs mapped to the expected QTL regions. The SNPs listed in Table 2 represent additional marker loci that can be used to select favorable QTL alleles (i.e. QTL alleles associated with enhanced ore newly conferred GLS resistance).

TABLE 2
SNP markers associated with GLS resistance QTLs
identified in the XR411 × JS891 RIL population.

| | | | | GLS | SNP |
|---|---|---|---|---|---|
| SEQ | | | GLS | suscep- | Position in |
| ID | | QTL | resistance | tibility | Reference |
| NO: | SNP marker name | name | allele | allele | Sequence |
| 1 | ZM_C4_183209964 | 4A | A | G | 61 |
| 2 | ZM_C4_183640675 | 4A | C | T | 51 |
| 3 | ZM_C4_189294989 | 4A | C | G | 61 |
| 4 | ZM_C4_187988553 | 4A | C | A | 51 |
| 5 | ZM_C9_124028957 | 9A | C | T | 61 |
| 6 | ZM_C9_125171993 | 9A | T | C | 51 |
| 7 | ZM_C9_125804907 | 9A | T | G | 61 |
| 8 | ZM_C9_126185898 | 9A | G | A | 51 |
| 9 | ZM_C9_126400936 | 9A | A | G | 51 |
| 10 | ZM_C9_126401198 | 9A | T | C | 51 |
| 39 | ZM_C9_127295062 | 9A | C | T | 51 |
| 40 | ZM_C9_131381146 | 9A | C | T | 61 |
| 41 | ZM_C9_131517485 | 9A | T | C | 51 |
| 42 | ZM_C9_130093144 | 9A | G | A | 51 |
| 43 | ZM_C9_128412180 | 9A | A | G | 61 |
| 44 | ZM_C9_131161648 | 9A | C | T | 51 |
| 45 | ZM_C9_129403817 | 9A | G | A | 61 |
| 11 | ZM_C9_139961409 | 9B | G | A | 51 |
| 12 | ZM_C9_142658967 | 9B | C | T | 51 |
| 13 | ZM_C9_151296063 | 9C | G | T | 61 |
| 14 | ZM_C9_151687245 | 9C | C | A | 51 |
| 46 | ZM_C9_152795210 | 9C | T | C | 51 |
| 15 | CPGR.00012 | 4A | C | T | 61 |
| 16 | CPGR.00015 | 4A | C | G | 61 |
| 17 | CPGR.00086 | 4A | A | C | 61 |
| 18 | CPGR.00090 | 4A | T | C | 61 |
| 19 | CPGR.00016 | 4A | C | T | 61 |
| 20 | CPGR.00038 | 4A | G | A | 61 |
| 21 | CPGR.00098 | 4A | G | A | 61 |
| 22 | CPGR.00102 | 4A | G | A | 61 |
| 23 | CPGR.00053 | 9B | C | T | 61 |
| 24 | CPGR.00125 | 9B | A | T | 61 |
| 25 | CPGR.00054 | 9B | T | G | 61 |
| 26 | CPGR.00127 | 9B | C | A | 61 |
| 27 | CPGR.00131 | 9B | A | T | 61 |
| 28 | CPGR.00120 | 9B | A | G | 61 |

### Example 7

### Identification of SSR Markers in the GLS QTL Regions

To identify SSR markers in the GLS QTL regions, SSR marker analysis of DNA extracted from each individual RIL in the maize population was carried out using methods known in the art (Taramino and Tingey. 1996. Genome 39:277-287). SSR markers were chosen based on their position between the flanking SNP markers of the GLS resistance QTL using bioinformatics methods known in the art. The PCR primers for the SSR analysis were obtained from the publicly available Maize Genetics and Genomics Database. Although the primers were obtained from this database, other suitable primers can be designed using any suitable method. The primers generate an amplified PCR product or marker locus or portion of the marker locus (markers) having at least 50 base pair in length. Individual plants of the maize RIL population were analyzed using the selected SSR markers. The SSR markers that map to the GLS resistance QTL regions were added to the list of marker loci that can be used in subsequent marker assisted breeding for GLS resistance, and are listed in Table 3.

Detection of markers (shown in Tables 2 and 3) in the QTL regions can be used in marker-assisted maize breeding programs to develop maize plants carrying one or more of the favorable QTL alleles (i.e. the QTL alleles associated with newly conferred or enhanced resistance to gray leaf spot), namely QTL4A, QTL9A, QTL9B and/or QTL9C.

TABLE 3
SSR markers that can be used to identify plants that
contain GLS resistance QTL4 or 9 or to counterselect
against the GLS susceptible alleles of these QTL.

| | | | | SSR marker | SSR marker |
|---|---|---|---|---|---|
| | | | | locus size | locus size |
| | | | | associated | associated |
| | SSR | | | with GLS | with GLS |
| QTL | marker | Forward | Reverse | resistance | susceptibility |
| name | name | Primer | Primer | (bp) | (bp) |
| 4A | bnlg1927 | SEQ ID | SEQ ID | 192 | 207 |
| | | NO: 29 | NO: 30 | | |
| 4A | mmc0321 | SEQ ID | SEQ ID | 123 | 125 |
| | | NO: 31 | NO: 32 | | |
| 9B | umc1733 | SEQ ID | SEQ ID | 78 | 70 |
| | | NO: 33 | NO: 34 | | |
| 9B | bnlg1191 | SEQ ID | SEQ ID | 216 | 230 |
| | | NO: 35 | NO: 36 | | |
| 9B | umc1675 | SEQ ID | SEQ ID | 155 | 162 |
| | | NO: 37 | NO: 38 | | |

### Example 8

### Introgressing the GLS Resistance QTL Allele into Another Maize Background

To introduce the GLS resistance QTL allele into another maize inbred line, the donor line that contains the favorable QTL allele will be crossed with the inbred line (e.g. B73). To confirm the presence of the favorable QTL allele in the inbred line, markers such as but not limited to the SNP and/or SSR markers provided in Tables 2 and 3 can be used to perform initial screening of progeny plants. These markers will confirm the presence of the favorable QTL allele in the inbred line. In further crosses, marker analysis can be used to select the progeny maize lines with the favorable QTL allele.

As an example, the favorable allele at QTL4A was introgressed into the B73 and Mo17 backgrounds. The presence of the favorable QTL allele was confirmed with markers, and the % donor background in both inbred lines were calculated to be low. The inbred lines with the favorable QTL allele were grown in the field together with inbred control lines containing not containing the favorable QTL allele and the GLS disease levels in all lines were assessed. Both sets of inbred lines with the favorable QTL allele (B73+QTL and Mo17+QTL) showed significantly lower levels of GLS disease compared to their control lines (B73 and Mo17, respectively) (Table 4). GLS disease levels are expressed as average Area Under Disease Progress Curve (AUDPC) which is a useful quantitative measure of disease severity over time.

TABLE 4
GLS disease scores expressed as average Area Under Disease
Progress Curve (AUDPC) of B73 and Mo17 inbred lines
with the favorable QTL4A allele introgressed.

| | | | Significantly |
|---|---|---|---|
| | | | different |
| | | Standard | compared to |
| Background | Average AUDPC | Deviation | control * |
| B73 control | 206.00 | ±11.76 | N/A |
| B73 + QTL | 165.70 | ±10.42 | Yes |
| Mo17 control | 126.30 | ±7.29 | N/A |
| Mo17 + QTL | 98.00 | ±9.00 | Yes |

* Significance based on Student's t-test, P < 0.01).

### Example 9

### Characterization of QTL Intervals and Haplotype Identification

The current physical map positions of the markers listed in Tables 2 and 3 were determined in order to place the markers in order and define the endpoints of the QTL intervals. Table 5 provides the markers and their positions on the B73 reference map. Hence QTL4A can be defined by and includes markers bnlg1927 and CPGR.00012; QTL9A can be defined by and includes markers ZM_C9_124028957 and ZM_C9_131517485; QTL9B can be defined by and includes markers CPGR.00127 and CPGR.00054; and QTL9C can be defined by and includes markers umc1675 and ZM_C9_52795210.

TABLE 5
Markers and their current positions on the B73 reference genome

| | | Physical position in bp based |
|---|---|---|
| | | on B73 RefGen_v2 genome |
| Marker name | QTL name | sequence |
| bnlg1927 | 4A | 180, 440, 879 |
| ZM_C4_183209964 | 4A | 183, 209, 964 |
| ZM_C4_183640675 | 4A | 183, 640, 675 |
| CPGR.00086 | 4A | 186, 589, 176 |
| ZM_C4_187988553 | 4A | 187, 988, 553 |
| ZM_C4_189294989 | 4A | 189, 294, 989 |
| mmc0321 | 4A | 190, 336, 170 |
| CPGR.00102 | 4A | 197, 370, 063 |
| CPGR.00038 | 4A | 207, 855, 347 |
| CPGR.00015 | 4A | 212, 750, 962 |
| CPGR.00090 | 4A | 219, 602, 640 |
| CPGR.00098 | 4A | 221, 759, 681 |
| CPGR.00016 | 4A | 229, 409, 034 |
| CPGR.00012 | 4A | 231, 730, 671 |
| ZM_C9_124028957 | 9A | 124, 028, 957 |
| ZM_C9_125171993 | 9A | 125, 171, 993 |
| ZM_C9_125804907 | 9A | 125, 804, 907 |
| ZM_C9_126185898 | 9A | 126, 185, 898 |
| ZM_C9_126400936 | 9A | 126, 400, 936 |
| ZM_C9_126401198 | 9A | 126, 401, 198 |
| ZM_C9_127295062 | 9A | 127, 295, 062 |
| ZM_C9_128412180 | 9A | 128, 412, 180 |
| ZM_C9_129403817 | 9A | 129, 403, 817 |
| ZM_C9_130093144 | 9A | 130, 093, 144 |
| ZM_C9_131161648 | 9A | 131, 161, 648 |
| ZM_C9_131381146 | 9A | 131, 381, 146 |
| ZM_C9_131517485 | 9A | 131, 517, 485 |
| CPGR.00127 | 9B | 138, 754, 340 |
| ZM_C9_139961409 | 9B | 139, 961, 409 |
| CPGR.00131 | 9B | 141, 937, 953 |
| ZM_C9_142658967 | 9B | 142, 658, 967 |
| bnlg1191 | 9B | 144, 922, 472 |
| CPGR.00125 | 9B | 145, 041, 508 |
| umc1733 | 9B | 145, 339, 729 |
| CPGR.00120 | 9B | 145, 588, 407 |
| CPGR.00053 | 9B | 146, 467, 205 |
| CPGR.00054 | 9B | 146, 467, 696 |
| umc1675 | 9C | 149, 252, 474 |
| ZM_C9_151296063 | 9C | 151, 296, 063 |
| ZM_C9_151687245 | 9C | 151, 687, 245 |
| ZM_C9_152795210 | 9C | 152, 795, 210 |

The markers in each QTL interval with the highest LOD scores in each test allowed the identification of favorable haplotypes (i.e. haplotypes associated with newly conferred or enhanced resistance to gray leaf spot). Favorable haplotypes at QTL4A include a “C” at ZM_C4_183640675; a “C” at ZM_C4_187988553; and a “C” at ZM_C4_189294989. Favorable haplotypes at QTL9A include a “T” at ZM_C9_125804907; a “G” at ZM_C9_126185898; an “A” at ZM_C9_126400936; and a “T” at ZM_C9_126401198. Favorable haplotypes at QTL9B include a “G” at ZM_C9_139961409 and a “C” at ZM_C9_142658967; Favorable haplotypes at QTL9C include a “G” at ZM_C9_151296063; a “C” at ZM_C9_151687245; and a “T” at ZM_C9_152795210.

What is claimed is:

1. A method of identifying and/or selecting a maize plant that displays newly conferred or enhanced resistance to gray leaf spot caused by Cercospora spp., wherein said method comprises:
a. detecting the presence of at least one allele of a marker locus in the DNA of a maize plant wherein said marker locus is located within QTL4A, QTL9A, QTL9B, or QTL9C, and said allele is associated with newly conferred or enhanced resistance to gray leaf spot; and
b. selecting the maize plant that has the allele associated with newly conferred or enhanced resistance to gray leaf spot.

2. The method of claim 1, wherein said gray leaf spot is caused by Cercospora zeina.

3. The method of claim 1, wherein QTL4A is defined by and includes markers bnlg1927 and CPGR.00012.

4. The method of claim 3, wherein the at least one allele of the marker locus is associated with one or more of the following:
a. a product of 192 bp in size when amplified with primers having SEQ ID NOs:29 and 30;
b. an “A” at ZM_C4_183209964;
c. a “C” at ZM_C4_183640675;
d. a “C” at ZM_C4_189294989;
e. a “C” at ZM_C4_187988553;
f. a “C” at CPGR.00012;
g. a “C” at CPGR.00015;
h. an “A” at CPGR.00086;
i. a “T” at CPGR.00090;
i. a “C” at CPGR.00016;
k. a “G” at CPGR.00038;
l. a “G” at CPGR.00098;
m. a product of 123 bp in size when amplified with primers having SEQ ID NOs:31 and 32; and
n. a “G” at CPGR.00102.

5. The method of claim 1, wherein QTL9A is defined by and includes markers ZM_C9_124028957 and ZM_C9_131517485.

6. The method of claim 5, wherein the at least one allele of the marker locus is associated with one or more of the following:
a. a “C” at ZM_C9_124028957;
b. a “T” at ZM_C9_125171993;
c. a “T” at ZM_C9_25804907;
d. a “G” at ZM_C9_126185898;
e. an “A” at ZM_C9_126400936;
f. a “T” at ZM_C9_126401198;
g. a “C” at ZM_C9_127295062;
h. a “C” at ZM_C9_131381146;
i. a “T” at ZM_C9_31517485;
j. a “G” at ZM_C9_130093144;
k. an “A” at ZM_C9_128412180;
l. a “C” at ZM_C9_131161648; and
m. a “G” at ZM_C9_29403817.

7. The method of claim 1, wherein said QTL9B is defined by and includes markers CPGR.00127 and CPGR.00054.

8. The method of claim 7, wherein the at least one allele of the marker locus is associated with one or more of the following:
a. a “G” at ZM_C9_39961409;
b. a “C” at ZM_C9_142658967;
c. a “C” at CPGR.00053;
d. an “A” at CPGR.00125;
e. a “T” at CPGR.00054;
f. a “C” at CPGR.00127;
g. an “A” at CPGR.00131;
h. an “A” at CPGR.00120;
i. a product of 216 bp in size when amplified with primers having SEQ ID NOs:35 and 36; and
j. a product of 78 bp in size when amplified with primers having SEQ ID NOs:33 and 34.

9. The method of claim 1, wherein said QTL9C is defined by and includes markers umc1675 and ZM_C9_152795210.

10. The method of claim 9, wherein the at least one allele of the marker locus is associated with one or more of the following:
a. a product of 155 bp in size when amplified with primers having SEQ ID NOs:37 and 38;
b. a “G” at ZM_C9_151296063;
c. a “C” at ZM_C9_151687245; and
d. a “T” at ZM_C9_52795210.

11. A method of introgressing a QTL allele associated with newly conferred or enhanced resistance to gray leaf spot caused by Cercospora spp. into a maize plant, said method comprising:
a. crossing a first maize plant comprising a QTL allele associated with newly conferred or enhanced resistance to gray leaf spot with a second maize plant to obtain a population of progeny plants;
b. screening the progeny plants with at least one marker located within 10 cM of any of the group consisting of:
i. ZM_C4_183209964;
ii. ZM_C4_183640675;
iii. ZM_C4_189294989;
iv. ZM_C4_187988553;
v. ZM_C9_124028957;
vi. ZM_C9_125171993;
vii. ZM_C9_125804907;
viii. ZM_C9_126185898;
ix. ZM_C9_126400936;
x. ZM_C9_126401198;
xi. ZM_C9_127295062;
xii. ZM_C9_131381146;
xiii. ZM_C9_131517485;
xiv. ZM_C9_130093144;
xv. ZM_C9_28412180;
xvi. ZM_C9_31161648;
xvii. ZM_C9_29403817;
xviii. ZM_C9_39961409;
xix. ZM_C9_42658967;
xx. ZM_C9_51296063;
xxi. ZM_C9_51687245;
xxii. ZM_C9_52795210;
xxiii. CPGR.00012;
xxiv. CPGR.00015;
xxv. CPGR.00086;
xxvi. CPGR.00090;
xxvii. CPGR.00016;
xxviii. CPGR.00038;
xxix. CPGR.00098;
xxx. CPGR.00102;
xxxi. CPGR.00053;
xxxii. CPGR.00125;
xxxiii. CPGR.00054;
xxxiv. CPGR.00127;
xxxv. CPGR.00131;
xxxvi. CPGR.00120;
xxxvii. bnlg1927;
xxxviii. mmc0321;
xxxix. umc1733;
xl. bnlg1191; and
xli. umc1675; wherein said marker comprises an allele associated with newly conferred or enhanced resistance to gray leaf spot; and
c. determining if the progeny plants comprise the QTL allele associated with newly conferred or enhanced resistance to gray leaf spot.

12. The method of claim 11, wherein said gray leaf spot is caused by Cercospora zeina.

13. A method of identifying a maize plant containing at least one allele of a marker locus associated with newly conferred or enhanced resistance to gray leaf spot caused by Cercospora spp., wherein said method comprises:
a. genotyping at least one maize plant with at least one marker wherein said marker is linked to a member of the group consisting of:
i. ZM_C4_183209964;
ii. ZM_C4_183640675;
iii. ZM_C4_189294989;
iv. ZM_C4_187988553;
v. ZM_C9_24028957;
vi. ZM_C9_125171993;
vii. ZM_C9_25804907;
viii. ZM_C9_26185898;
ix. ZM_C9_26400936;
x. ZM_C9_126401198;
xi. ZM_C9_27295062;
xii. ZM_C9_31381146;
xiii. ZM_C9_31517485;
xiv. ZM_C9_130093144;
xv. ZM_C9_28412180;
xvi. ZM_C9_31161648;
xvii. ZM_C9_29403817;
xviii. ZM_C9_39961409;
xix. ZM_C9_42658967;
xx. ZM_C9_51296063;
xxi. ZM_C9_51687245;
xxii. ZM_C9_52795210;
xxiii. CPGR.00012;
xxiv. CPGR.00015;
xxv. CPGR.00086;
xxvi. CPGR.00090;
xxvii. CPGR.00016;
xxviii. CPGR.00038;
xxix. CPGR.00098;
xxx. CPGR.00102;
xxxi. CPGR.00053;
xxxii. CPGR.00125;
xxxiii. CPGR.00054;
xxxiv. CPGR.00127;
xxxv. CPGR.00131;
xxxvi. CPGR.00120;
xxxvii. bnlg1927;
xxxviii. mmc0321;
xxxix. umc1733;
xl. bnlg1191; and
xli. umc1675; and
b. selecting a maize plant containing at least one allele at the marker that is associated with newly conferred or enhanced resistance to gray leaf spot.

14. The method of claim 13, wherein said gray leaf spot is caused by Cercospora zeina.

15. The method of claim 13, wherein said marker locus is linked to any of the markers in the group consisting of (i)-(xli) by 10 cM on a single meiosis based genetic map.

16. The method of claim 13, wherein said marker locus is linked to any of the markers in the group consisting of (i)-(xli) by 5 cM on a single meiosis based genetic map.

17. The method of claim 13, wherein said marker locus is linked to any of the markers in the group consisting of (i)-(xli) by 1 cM on a single meiosis based genetic map.

18. A method of identifying and/or selecting a maize plant with newly conferred or enhanced resistance to gray leaf spot caused by Cercospora spp., said method comprising:
a. detecting in a maize plant at least one marker allele that is linked to and associated with:
i. a haplotype comprising: a “C” at ZM_C4_183640675; a “C” at ZM_C4_187988553; and a “C” at ZM_C4_189294989;
ii. a haplotype comprising: a “T” at ZM_C9_125804907; a “G” at ZM_C9_26185898; an “A” at ZM_C9_26400936; and a “T” at ZM_C9_126401198;
iii. a haplotype comprising: a “G” at ZM_C9_139961409 and a “C” at ZM_C9_42658967; or
iv. a haplotype comprising: a “G” at ZM_C9_151296063; a “C” at ZM_C9_51687245; and a “T” at ZM_C9_52795210; and
b. selecting said maize plant having the at least one marker allele.

19. The method of claim 18, wherein said gray leaf spot is caused by Cercospora zeina.

20. The method of claim 18, wherein said at least one marker allele is linked to the haplotype in (i) or (ii) by 10 cM on a single meiosis based genetic map.

21. The method of claim 18, wherein said at least one marker allele is linked to the haplotype in (i) or (ii) by 5 cM on a single meiosis based genetic map.

22. The method of claim 18, wherein said at least one marker allele is linked to the haplotype in (i) or (ii) by 1 cM on a single meiosis based genetic map.


SEQUENCE LISTING (46 sequences)

SEQ ID NO: 1 - DNA, 121 bp, Artificial
  ZM_C4_183209964 Reference sequence
gtgtatgcaaaattcacagcaaatatcatggccagcatggtatcacgatatccacaggaargttgggcacaagagggtggtgatccgtgcttaccagcatcatggtcctgggtgcaatgca

SEQ ID NO: 2 - DNA, 101 bp, Artificial
  ZM_C4_183640675 Reference sequence
gccctgttagatgtaaacaaatagtacactgatcccattaataagtgatgyacagacagacccttgcttttctccttctgtctgtctctgcactgcacggg

SEQ ID NO: 3 - DNA, 121 bp, Artificial
  ZM_C4_189294989 Reference sequence
gaagcttcgaaattctgactgctggctgccatcgggacaacaagcgtctnccaagatgtgscgtagcttcctggccttgccgacaccctgtggcagcctctgtactctggtttgcctgacg

SEQ ID NO: 4 - DNA, 101 bp, Artificial
  ZM_C4_187988553 Reference sequence
actttttttcataaaagagaagtatcaaatatgtgacatcaaagtaataamgtatgatgtgagacgacaaaaagttcccgggtgtgaaaacagacctagaa

SEQ ID NO: 5 - DNA, 121 bp, Artificial
  ZM_C9_124028957 Reference sequence
tgtttttttggcttttctagacacataactcttgcaatgcactaaaaggtgagagcagctycagtgttggtgaaaatcgnatcatggaaagagcaaatagaaccgtaaacactagaggaag

SEQ ID NO: 6 - DNA, 101 bp, Artificial
  ZM_C9_125171993 Reference sequence
atgacatgtcaaggtactgaatctacttgtcctgatcttttctctcttgcyctgccagtaccatgcatctagtttccatgatcttcagcgaggaggctgtg

SEQ ID NO: 7 - DNA, 121 bp, Artificial
  ZM_C9_125804907 Reference sequence
tgactggtaaagtcaaattcaacgacggatcgtatttaactatgagggtcgttgtcgttckcgagatgttcaaaagatagtatgtttcatgtctacgttgtagatagtacgtttgggacac

SEQ ID NO: 8 - DNA, 101 bp, Artificial
  ZM_C9_126185898 Reference sequence
gtttatctgcgtggagatattagggagtgtactgatgtatatgtacaggarggagggcagcgtgattcagaacaggattttatattgcagagaagggcgtg

SEQ ID NO: 9 - DNA, 101 bp, Artificial
  ZM_C9_126400936 Reference sequence
tgagatagagcagcgaggttgatgacaagcctgttggatctggttttctargccactttcgattttttatcgagcaaatatcctttaacgagtctgttgga

SEQ ID NO: 10 - DNA, 101 bp, Artificial
  ZM_C9_126401198 Reference sequence
ccagtgtctattacgagggggcctgttgcaacacaactgagcgacataggytgtgttttcggcagcgagcgtgaatatagtcgtacaaaacattgttttgt

SEQ ID NO: 11 - DNA, 101 bp, Artificial
  ZM_C9_139961409 Reference sequence
aacggtaggaaattttcttcaaaaaatagcagttagtatactactagagcraaagctgcaaccaaacttgtaggatccatctggacatgtgttgtatccaa

SEQ ID NO: 12 - DNA, 101 bp, Artificial
  ZM_C9_142658967 Reference sequence
aagcaactcgagcgtagcttcaacagctcactgacaaaattgcagatcaayttccaggatgtcctggaaaggcggatctgatgagtcgggcaggaagggcc

SEQ ID NO: 13 - DNA, 121 bp, Artificial
  ZM_C9_151296063 Reference sequence
ggccattttcctgaagaggttggcagcaccacaagtccacctgtgctgttgatgacggtakgccttgaaagtacttgggagttcactcttaacctgtacaaaaaactgttacaacagcaga

SEQ ID NO: 14 - DNA, 101 bp, Artificial
  ZM_C9_151687245 Reference sequence
tacaggacttgaagggtgtgcccggcctcgcatttaacagttatcagtcgmttgcatcattccgacaatttgtttttgtgtgtggttattttggttgtcgt

SEQ ID NO: 15 - DNA, 121 bp, Artificial
  CPGR.00012 Reference sequence
tttgacttctgttttgatttcctatcacgcttcctacgccttctttcacgcctttggtcaygctttcttcttgaacgcttgcgtttatttttcttcgaatgtctctttctgcgccttcgcc

SEQ ID NO: 16 - DNA, 121 bp, Artificial
  CPGR.00015 Reference sequence
attcctatttttactttgcatcaaattgaccatgtatttatcatcaacagatgcaattctstcgttgaagttagaggatagctctttaccaactgcccctgcacttgtactattatctgaa

SEQ ID NO: 17 - DNA, 121 bp, Artificial
  CPGR.00086 Reference sequence
cagcaagcgggctccgtggctgccgtcgattccatcggttcccccgcctcctcggctcctmgattttcacgattcttccgctgtcggtcttggggtgggggatgacggagggggagagcgg

SEQ ID NO: 18 - DNA, 121 bp, Artificial
  CPGR.00090 Reference sequence
acctgagtgttccaagtaccatgacgtgcctgctgtgatctttgctctgactggatacacyggaaatctattccatgacttcaccgacgtgcttgtcccccttttcacgactgcaagcgag

SEQ ID NO: 19 - DNA, 121 bp, Artificial
  CPGR.00016 Reference sequence
cctggagatgcctgactggcccctccaggaccaccatagttggacccatcctgggtcgagyttgcagctgatccataagcagatgtgccataaccctgctgatcataaccagtctgctgag

SEQ ID NO: 20 - DNA, 121 bp, Artificial
  CPGR.00038 Reference sequence
tcccctatcctgcactagaattgtttccggaggatgatctcagcaactgatggtggttagragcaatcgaattgggcatgccagagttgaacatgtttgatgattgtggtgatggttgagt

SEQ ID NO: 21 - DNA, 121 bp, Artificial
  CPGR.00098 Reference sequence
gcatgctgggccttgcactggcaaacgggtgtgagcacaacagtcccagcttcaacgccargcacgcctcgtcgacatcgtagtcccctttgagcctgccgtcgatcgcatcggtgagcga

SEQ ID NO: 22 - DNA, 121 bp, Artificial
  CPGR.00102 Reference sequence
ttcagatggcagcttctttatggacagtaagaacttcttcagatgtcgaatgaacactggraatttctccaggttgccatggtcacctccctcgatccacaggggttcgtacttgtgctgg

SEQ ID NO: 23 - DNA, 121 bp, Artificial
  CPGR.00053 Reference sequence
tgtagcacttcttgctgcatgagaggtgaaatgttctttccaacctcaatagcagcattgycagtcttgtgtaatgccgtcacttcttcctgcactgcttgactaaaattggatgaagcac

SEQ ID NO: 24 - DNA, 121 bp, Artificial
  CPGR.00125 Reference sequence
cttcttgaacagagataccttggttggcgtacttcttcttattgatgccagtattccaccwcagaaaattgatctagactgtgctaactggctcggtcgtaacaatgtaaggcagtatgca

SEQ ID NO: 25 - DNA, 121 bp, Artificial
  CPGR.00054 Reference sequence
tccattagctcgagctcccaaatttgcttggaccccagaattcagtttcttcgccttaccktgctccagtcccttctcctttacacgacttgactcaacctgcttccgatgctccacggca

SEQ ID NO: 26 - DNA, 121 bp, Artificial
  CPGR.00127 Reference sequence
tcgatcgatctatatttgttcagctaatttcaaaacatttgaccaaccttcgttttgaacmtccagattgcttaaagcctgtgaccaaggacgagactttcgccagaagcaccagcgtagt

SEQ ID NO: 27 - DNA, 121 bp, Artificial
  CPGR.00131 Reference sequence
ttagattatttctgcatttgaccactaatattaaaggttttgattacatagacggcccggwcagagcagaataacctccacaaggaaggagactgatacatgcaaaattctgtcagggaca

SEQ ID NO: 28 - DNA, 121 bp, Artificial
  CPGR.00120 Reference sequence
gagcttcttgggatgaagagtccaactgcatttatcaagcgaaaaactgaacgaacaaaarcatccatcctccaaggaaaagaagcagggccctgcatcaatttcattatccatgtatttc

SEQ ID NO: 29 - DNA, 18 bp, Artificial
  bnlg1927 forward primer
ctgacgattcatgtggct

SEQ ID NO: 30 - DNA, 20 bp, Artificial
  bnlg1927 reverse primer
tcctagtacaccgcaactct

SEQ ID NO: 31 - DNA, 17 bp, Artificial
  mmc0321 forward primer
taatccgagctgcagag

SEQ ID NO: 32 - DNA, 18 bp, Artificial
  mmc0321 reverse primer
cacatgagctgagcatca

SEQ ID NO: 33 - DNA, 23 bp, Artificial
  umc1733 forward primer
acacccaacctcccactgtaaaa

SEQ ID NO: 34 - DNA, 22 bp, Artificial
  umc1733 reverse primer
gattgggattgggattggaaat

SEQ ID NO: 35 - DNA, 20 bp, Artificial
  bnlg1191 forward primer
aatcatgcgtaggcgtagct

SEQ ID NO: 36 - DNA, 20 bp, Artificial
  bnlg1191 reverse primer
gccagaggaaaaagaaggct

SEQ ID NO: 37 - DNA, 24 bp, Artificial
  umc1675 forward primer
gttcttcctcttccccatcagtct

SEQ ID NO: 38 - DNA, 21 bp, Artificial
  umc1675 reverse primer
atagctgcgcgtaaagcaacc

SEQ ID NO: 39 - DNA, 101 bp, Artificial
  ZM_C9_127295062 Reference sequence
agtattagctgctcgcaccatcgctgtgccatctgttccgtagcttccgcygtcactgccgacatgctctgaactcgagaccgacagcaggaacaatgtca

SEQ ID NO: 40 - DNA, 121 bp, Artificial
  ZM_C9_131381146 Reference sequence
actcaccatctaaattcatggtttggatataagctataccatttggatcgctcgtacccaynagaaccatgtcacacggcatggtctcattggcatgaattttgacaacctcccctgcaca

SEQ ID NO: 41 - DNA, 100 bp, Artificial
  ZM_C9_131517485 Reference sequence
ttggtaatcattcatcacggacttcacaaggacacatcaattatgtgttcycacaatcttttgggtccggttgagcgaaatggacggcaaattatatgtt

SEQ ID NO: 42 - DNA, 101 bp, Artificial
  ZM_C9_130093144 Reference sequence
cgtcaggagctgcggcggcccctgtcgttcaggtgagcgtccatggctttrtcacctgccgccgagtactcgaatgctcttcgatcccataccatcatatg

SEQ ID NO: 43 - DNA, 121 bp, Artificial
  ZM_C9_128412180 Reference sequence
tggagaaagaaatttggtcatcaaatggttccgaggaggagaaacttcaaaatcttctgcrttgttggattagttggatgttttctgatatagtgataccattagtccagtcatattttta

SEQ ID NO: 44 - DNA, 101 bp, Artificial
  ZM_C9_131161648 Reference sequence
ttcagatatatgccttttctcaaaggccggcagatctaggggtataacttytctcgaacactggcagatccttgttacacaatgattgatagcattggaca

SEQ ID NO: 45 - DNA, 121 bp, Artificial
  ZM_C9_129403817 Reference sequence
gtcataacccaagatggcgtcatacggtcccagatccaacactctcatatcagtgtggaargtgtggccatttgcgcaccaggcaagctgaggaaccaacaaatccgacgttagggactcg

SEQ ID NO: 46 - DNA, 101 bp, Artificial
  ZM_C9_152795210 Reference sequence
acttcatcatctgaggctagtcaacggaaaaaggtttgacatacgtcctgyaaaaaaatagttaacatgaacttgaggatctgctgcagtccaggaaagtt